(19) United States
(12) Patent Application Publication
(10) Pub. No.: US 2016/0367976 A1
(43) Pub. Date: Dec. 22, 2016

(54) IONIC FLUID PRECURSORS

(71) Applicant: RELIANCE INDUSTRIES LIMITED, Mumbai (IN)

(72) Inventors: Parasu Veera UPPARA, Navi Mumbai (IN); Pavankumar ADURI, Thane (IN); Subarna SHYAMROY, Kolkata (IN)

(73) Assignee: RELIANCE INDUSTRIES LIMITED, Mumbai (IN)

(21) Appl. No.: 15/256,988

(22) Filed: Sep. 6, 2016

Related U.S. Application Data
(63) Continuation of application No. PCT/IB2015/051508, filed on Mar. 2, 2015.

(30) Foreign Application Priority Data
Mar. 4, 2014 (IN) 729/MUM/2014

(51) Int. Cl. B01J 31/04; B01J 35/12; C07C 51/41; C07C 309/30; C07C 303/22; C07C 53/10; B01J 31/02; C09K 3/00

(52) U.S. Cl. CPC B01J 31/04; B01J 31/0289; B01J 35/12; C09K 3/00; C07C 309/30; C07C 303/22; C07C 53/10; C07C 51/418; C07C 51/412

(57) ABSTRACT

The present disclosure provides an ionic fluid pre-cursor being a reaction product of at least one compound of formula (I) and at least one hydrogen donor and having a softening point less than the melting point or softening point of said compound of formula (I)

MₓA_{y}.zH₂O

The present disclosure also provides a process for preparing the ionic fluid pre-cursor. The present disclosure further provides an ionic fluid and a process for preparing the same.

### CROSS-REFERENCE TO RELATED APPLICATIONS

This application is a continuation of PCT Application No. PCT/IB2015/051508, filed on Mar. 2, 2015, which claims the benefit of Indian Patent Application No. 729/MUM/2014, filed on Mar. 4, 2014. The entire disclosures of each of the above applications are incorporated herein by reference.

### FIELD OF THE DISCLOSURE

The present disclosure relates to precursors of ionic fluid/liquid and processes for preparation thereof. The present disclosure also relates to a process for the preparation of ionic fluid/liquid.

### BACKGROUND

Ionic compositions are compounds in which ions are held together in a lattice structure by ionic bonds. Ionic compositions have high melting and boiling points and exhibit very low or no vapor pressure. The afore-stated properties render them innocuous from human health and environment point of view. Ionic compositions find multifarious applications in fields such as synthetic chemistry, electrochemistry, pyrolysis and gasification.

Over the years many methods have been devised for the preparation of ionic liquids. U.S. Pat. No. 4,764,440 suggests low temperature molten compositions, obtained by reacting, for example, trimethylphenylammonium chloride with aluminum trichloride at 45° C. The resulting ionic composition has a low freezing point (around −75° C.); however, said composition has a drawback of water sensitivity because of the presence of aluminum trichloride.

Another U.S. Pat. No. 5,731,101 suggests a process for forming a low temperature molten ionic liquid composition by mixing metal halides such as aluminum halide, gallium halide, iron halide, copper halide, zinc halide, and indium halide and an alkyl-containing amine hydrohalide salt. Particularly, aluminum trichloride and ferric trichloride are employed as metal halides. The metal halides form anion containing polyatomic chloride bridge in the presence of the alkyl-containing amine hydrohalide salt. However, the process disclosed in U.S. Pat. No. 5,731,101 has a limitation in that it cannot be applied for the preparation of ionic liquids containing metal halides other that the metal halides mentioned above. For instance, a low temperature molten ionic liquid composition containing tin halide or nickel halide cannot be prepared by the process disclosed in U.S. Pat. No. 5,731,101.

Still another U.S. Pat. No. 6,573,405 suggests a method for preparing an ionic compound by reacting a quaternary ammonium compound of the formula R¹R²R³R⁴N⁺X⁻ with a halide of zinc, tin or iron. However, the reaction is carried out at a temperature higher than 100° C. rendering the process energy inefficient.

Yet another U.S. Pat. No. 7,183,433 suggests a method of preparing an ionic compound having a freezing point of up to 100° C. by reacting amine salt of the formula R¹R²R³R⁴N⁺X⁻ with organic compound (II). U.S. Pat. No. 7,183,433 teaches that such types of reactions are generally endothermic and are usually carried out by heating to a temperature of at least 100° C. Particularly, U.S. Pat. No. 7,183,433 suggests the reaction of choline chloride and organic compounds such as urea, oxalic acid and malonic acid at a temperature of 70° C. The reaction is energy inefficient as it is carried out at a high temperature.

U.S. Pat. No. 7,196,221 discloses a method for preparing an ionic compound by reacting a quaternary ammonium compound of formula R¹R²R³R⁴N⁺X⁻ with a hydrated metal salt, which is a chloride, nitrate, sulphate or acetate of Li, Mg, Ca, Cr, Mn, Fe, Co, Ni, Cu, Zn, Cd, Pb, Bi, La Sn or Ce. The reaction for the preparation of ionic compound is carried out at a temperature of 120° C.

US Patent Publication No. 20090247432 suggests a process for reacting a quaternary ammonium chloride such as choline chloride and a hydrogen donor such as urea. The reaction comprises combining the quaternary ammonium chloride and the hydrogen donor to form a mixture followed by heating the mixture to a temperature greater than 70° C. to obtain an ionic liquid.

The drawback associated with these prior art processes is that they are carried out at a high temperature, making them energy inefficient and thus, uneconomical.

Accordingly, there is felt a need for a simple and energy efficient process for the preparation of ionic fluid precursors and ionic fluids. The present disclosure also envisages an ionic fluid precursor which exhibits a softening point less than 150° C. and which can be converted to ionic fluid without precipitation of salt.

### OBJECTS

Some of the objects of the present disclosure are discussed herein below:

It is an object of the present disclosure to provide ionic fluid precursors.

It is an object of the present disclosure to provide a process for the preparation of ionic fluid precursors.

It is another object of the present disclosure to provide a cost-efficient and environment friendly process for the preparation of ionic fluid precursors.

It is still another object of the present disclosure to provide ionic fluids from ionic liquid precursors.

It is still another object of the present disclosure to provide a simple and energy efficient process for the preparation of ionic fluids.

It is a further an object of the present disclosure to ameliorate one or more problems of the prior art or to at least provide a useful alternative.

Other objects and advantages of the present disclosure will be more apparent from the following description which is not intended to limit the scope of the present disclosure.

### SUMMARY

The present disclosure provides an ionic fluid pre-cursor, being a reaction product of at least one compound of formula (I) and at least one hydrogen donor and having a softening point less than the melting point or softening point of said compound of formula (I)

MₓA_{y}.zH₂O Formula (I)
wherein,
M is independently selected from the group consisting of Na, K, Li, Mg, Ca, Cr, Mn, Fe, Co, Mo, Ni, Cu, Zn, Cd, Sn, Pb, St, Bi, La, Ce, Al, Hg, Cs, Rb, Sr, V, Pd, Zr, Au, Pt, quarternary ammonium, immidazolium, phosphonium, pyridinium and pyrrolidinium,
A is independently selected from the group consisting of Cl, Br, F, I, NO₃, SO₄, CH₃COO, HCOO and C₂O₄,
z is 0 to 20, and
x and y are integers independently ranging from 1 to 20.

The precursor is maintained at a temperature of not more than 40° C.

During the preparation or storage of said ionic liquid pre-cursor and its conversion to ionic fluid, acidic fumes are not liberated in the form of compound of formula HₓAy.

The ionic fluid pre-cursor is adapted to convert into ionic fluid without precipitation of salt.

The hydrogen donor can be at least one compound selected from the group consisting of toluene-4-sulphonic acid monohydrate, oxalic acid, maleic acid, citric acid and methane sulfonic acid.

The molar ratio of compound of formula (I) to said hydrogen donor ranges from 1:1 to 1:6.

The ionic fluid precursor is capable of delivering a clear liquid when deployed as a constituent of a mixture comprising said ionic fluid precursor and at least one liquid medium and maintained at a temperature in the range of 0° C. to 40° C.

In accordance with another aspect of the present disclosure there is also provided an ionic fluid comprising:
an ionic fluid pre-cursor, being a reaction product of at least one compound of formula (I) and at least one hydrogen donor and having a softening point less than the melting point or softening point of said compound of formula (I); and
at least one liquid medium.

The molar ratio of compound of formula (I) to said hydrogen donor ranges from 1:1 to 1:6 and the weight ratio of the ionic fluid precursor to said medium ranges from 1:0.1 to 1:50.

In accordance with still another aspect of the present disclosure there is provided a process for the preparation of an ionic fluid precursor having a softening point less than the melting point or softening point of said compound of formula (I); said process comprising mixing at least one compound of formula MₓA_{y}.zH₂O (I) at a pre-determined proportion with at least one hydrogen donor at a temperature in the range of 0° C. to 40° C., to obtain the precursor, wherein said ionic fluid precursor is capable of delivering a clear liquid when deployed as a constituent of a mixture comprising said ionic fluid precursor and at least one liquid medium and maintained at a temperature in the range of 0° C. to 40° C.

In accordance with another aspect of the present disclosure there is provided a process for the preparation of ionic fluid; said process comprising the following steps:
mixing at least one compound of formula MₓA_{y}.zH₂O (I) with at least one hydrogen donor at a temperature in the range of 0° C. to 40° C. to obtain an ionic fluid precursor; and
incorporating at least one medium to said ionic fluid precursor followed by mixing to obtain an ionic fluid.

Alternatively, the process for the preparation of ionic fluid comprises mixing a) at least one compound of formula MₓA_{y}.zH₂O (I); b) at least one hydrogen donor; and c) at least one medium at a temperature in the range of 0 to 40° C. to obtain an ionic fluid.

The molar ratio of the compound of formula (I) to said hydrogen donor ranges from1:1 to 1:6 and the weight ratio of the ionic fluid precursor to said medium ranges from 1:0.1 to 1:50.

The amount of the medium ranges from 1% to 30% of the total weight of the compound of formula (I) and hydrogen donor.

### DETAILED DESCRIPTION

The present disclosure provides an ionic fluid pre-cursor, a reaction product of at least one compound of formula (I) and at least one hydrogen donor. The ionic fluid pre-cursor of the present disclosure is characterized by the following features:
The ionic fluid pre-cursor has a softening point less than the melting point or softening point of the starting material (compound of formula (I)),
during the preparation or storage of the ionic liquid pre-cursor and its conversion to ionic fluid, acidic fumes are not liberated in the form of compound of formula HₓAy,
the ionic fluid pre-cursor of the present disclosure is adapted to convert into ionic fluid without precipitation of salt, and
the ionic fluid pre-cursor is capable of delivering a clear liquid when deployed as a constituent of a mixture containing said ionic fluid precursor and at least one liquid medium and maintained at a temperature in the range of 0° C. to 40° C.

The compound of formula (I) is represented by:

MₓA_{y}.zH₂O
wherein,
M is independently selected from the group consisting of Na, K, Li, Mg, Ca, Cr, Mn, Fe, Co, Mo, Ni, Cu, Zn, Cd, Sn, Pb, St, Bi, La, Ce, Al, Hg, Cs, Rb, Sr, V, Pd, Zr, Au, Pt, quarternary ammonium, immidazolium, phosphonium, pyridinium and pyrrolidinium,
A is independently selected from the group consisting of Cl, Br, F, I, NO₃, SO₄, CH₃COO, HCOO and C₂O₄,
z is 0 to 20, and
x and y are integers independently ranging from 1 to 20.

In accordance with the present disclosure the molar ratio of compound of formula (I) to the hydrogen donor is maintained from 1:1 to 1:6. The hydrogen donor employed in accordance with the present disclosure includes but is not limited to toluene-4-sulphonic acid monohydrate, oxalic acid, maleic acid, citric acid and methane sulfonic acid. The ionic fluid pre-cursor of the present disclosure is maintained at a temperature of not more than 40° C.

In accordance with another aspect, the present disclosure provides a simple and energy efficient process for the preparation of the ionic fluid precursor. The process involves mixing at least one compound of formula (I) with at least one hydrogen donor. The process of the present disclosure avoids the use of heat to prepare the ionic fluid precursor. Instead, the present disclosure is focused on providing a process which involves utilization of physical mixing or mixing using mechanical means. The mixing step in accordance with the present disclosure can be carried out by using at least one device which includes but is not limited to a planetary mixer, a ball mill, a rod mill, a pebble mill, a vibratory pebble mill, a screw mill, a hammer mill, a jet mill, a muller, an agitator, multiplicity of rotors, a single rotor, a single blade mixer, a multi-blade mixer, a vessel with single or multiple agitators, a vessel with at least one baffle, a vessel with at least one baffle and at least one agitator, a vessel with at least one baffle and at least one airjet, a vessel with at least one baffle, at least one agitator and at least one airjet, an ultrasound cavitator and a hydrodynamic cavitator.

In accordance with the present disclosure the process is carried out at a temperature in the range of 0° C. to 40° C. In another embodiment the process is carried out at a temperature ranging from 0° C. to 30° C.

The resultant ionic fluid precursor exhibits a melting point less than 150° C., preferably, below 125° C.

The present disclosure also provides an ionic fluid containing the ionic fluid pre-cursor of the present disclosure and at least one liquid medium. The liquid medium includes but is not limited to methanol, ethanol, propan-1-ol, propan-2-ol, 1-butanol, isobutanol, 2-butanol, tert-butanol, dichloromethane, tetrahydrofuran, methyl acetate, ethyl acetate, acetone, dimethylformamide, acetonitrile, dimethyl sulfoxide, formic acid, acetic acid, methyl ethyl ketone, dimethyl carbonate, diethyl ketone, acetic anhydride, acetone, tert-butyl methyl ether, diethyl amine, diethylene glycol, N,N-dimethylacetamide, diethylene glycol dimethyl ether, ethylene glycol dimethyl ether, ethylene glycol, glycerin, hexamethylphosphor amide, hexamethylphosphorous triamide, isoamyl alcohol, 2-methoxyethanol, 2-methoxyethyl acetate, 1-methyl-2-pyrrolidinone, nitromethane, propanoic acid, pyridine, hydrogen fluoride, hydrogen chloride and water. In accordance with the present disclosure the weight ratio of the compound of formula (I) to the medium is maintained from 1:0.1 to 1:50.

In accordance with still another aspect of the present disclosure there is also provided a process for the preparation of ionic fluid. The process involves the following steps:

In the first step, at least one compound of formula MₓA_{y}.zH₂O (I) and at least one hydrogen donor selected from the group consisting of toluene-4-sulphonic acid monohydrate, oxalic acid, maleic acid, citric acid and methane sulfonic acid are mixed at a temperature ranging from 0 to 40° C. to obtain an ionic fluid precursor. The molar ratio of the compound of formula (I) to said hydrogen donor is maintained from 1:1 to 1:6.

In the next step, at least one liquid medium selected from the group consisting of methanol, ethanol, propan-1-ol, propan-2-ol, 1-butanol, isobutanol, 2-butanol, tert-butanol, dichloromethane, tetrahydrofuran, methyl acetate, ethyl acetate, acetone, dimethylformamide, acetonitrile, dimethyl sulfoxide, formic acid, acetic acid, methyl ethyl ketone, dimethyl carbonate, diethyl ketone, acetic anhydride, acetone, tert-butyl methyl ether, diethyl amine, diethylene glycol, N,N-dimethylacetamide, diethylene glycol dimethyl ether, ethylene glycol dimethyl ether, ethylene glycol, glycerin, hexamethylphosphor amide, hexamethylphosphorous triamide, isoamyl alcohol, 2-methoxyethanol, 2-methoxyethyl acetate, 1-methyl-2-pyrrolidinone, nitromethane, propanoic acid, pyridine, hydrogen fluoride, hydrogen chloride and water is incorporated to the ionic fluid precursor followed by mixing to obtain an ionic fluid. The weight ratio of the formula (I) to the medium is maintained from 1:0.1 to 1:50 to form ionic fluid. The amount of the medium employed ranges from 1% to 30% of the total weight of the compound of formula (I) and hydrogen donor.

Alternatively, the process involves mixing at least one compound of formula MₓA_{y}.zH₂O (I), at least one hydrogen donor and at least one liquid medium together to obtain the ionic fluid. The process is carried out at a temperature ranging from 0° C. to 40° C. The molar ratio of the compound of formula (I) to the hydrogen donor ranges from 1:1 to 1:6, whereas the weight ratio of the compound of formula (I) to the medium ranges from 1:0.1 to 1:50.

The ionic fluid precursors and ionic fluids according to the present disclosure may be utilized for a wide variety of applications in chemical and electrochemical field. The particular applications include solubilizing various chemicals such as fatty acids, greases, oils, metals, metals oxides and complexes, cellulose and various organic solvents. The ionic fluid precursors and ionic fluids also are used in extraction and surface modification.

Ionic fluid precursors and ionic fluids of the present disclosure are also found to be useful as inert media, solvents, co-solvents, catalysts or chemical reagents in the wide range of temperatures. In other applications, fluid precursors and ionic fluids are found to be useful as co-solvent and catalyst where aqueous and non-aqueous polar solvents may be employed. In other application, fluid precursors and ionic fluids are found to be useful in pure form or dissolved form in aqueous media or non-aqueous media as catalyst or co-solvent for chemical reactions.

Ionic fluid precursors and ionic fluids are found to be useful as acid catalysts for chemical reactions in both liquid form and immobilized state.

Hereinafter, the present disclosure will be described in more detail with reference to the following Examples, but the scope of the present disclosure is not limited thereto.

### EXAMPLE 1

### Preparation of Ionic Fluid Precursor

1.7 kilograms of p-Toluenesulfonic acid and 0.58 kilograms of sodium chloride were charged into different Hoppers. From the hoppers both the solids were passed through a screw conveyer to a planetary mixer operating at 80 rpm followed by mixing at 30° C. to form an ionic fluid precursor which was a thick semisolid paste.

### EXAMPLE 2

### Preparation of Ionic Fluid Precursor

0.518 kilograms of p-Toluenesulfonic acid and 0.382 kilograms of choline chloride (compound of formula I) were charged into different hoppers. From the hoppers both the solids were passed through a screw conveyer to a planetary mixer, operating at 80 rpm followed by mixing at 0° C. to form ionic fluid precursor. The resultant ionic fluid precursor was a viscous liquid.

### EXAMPLE 3

### Preparation of Ionic Fluid

2.28 kilograms of ionic fluid precursor as prepared in example 1 was transferred to a stirring vessel. To this 1.7 kg of methanol was added at 30° C. followed by mixing to obtain an ionic fluid.

### EXAMPLE 4

### Preparation of Ionic Fluid

0.9 kilograms of ionic fluid precursor as prepared in example 2 was transferred to a stirring vessel. To this precursor 0.0518 kilograms of methanol was added at 25° C. followed by mixing to obtain an ionic fluid.

### EXAMPLES 5 TO 43

### Preparation of Ionic Fluid Precursors

p-Toluenesulfonic acid and different salts in an equivalent molar ratio were charged into different Hoppers (refer the Table 1 below). From the hoppers both the solids were passed through a screw conveyer to planetary mixer to form an ionic fluid precursor at 25° C.

TABLE 1
Hydrogen Donor: Toluene-4-sulphonic acid monohydrate

| | | State of resultant |
|---|---|---|
| Example | Salt (melting point ° C.) | precursor (at ° C.) |
| | Chlorides | |
| 5 | Zinc Chloride (292° C.) | Semi Solid at 70 |
| 6 | Ferric Chloride (306° C.) | Semi Solid at 81 |
| 7 | Cobaltous Chloride (735° C.) | Semi Solid at 75 |
| 8 | Cuprous Chloride (426° C.) | Semi Solid at 67 |
| 9 | Mangenous Chloride (58° C.) | Semi Solid at 69 |
| 10 | Nickel Chloride (140° C.) | Semi Solid at 60 |
| 11 | Potassium Chloride (770° C.) | Semi Solid at 85 |
| 12 | Stannous Chloride (247° C.) | Semi Solid at 74 |
| 13 | Cesium Chloride (645° C.) | Semi Solid at 65 |
| 14 | Mercury Chloride (276° C.) | Semi Solid at 84 |
| | Fluorides | |
| 15 | Sodium Fluoride (993° C.) | Semi Solid at 105 |
| 16 | Potassium Fluoride (858° C.) | Semi Solid at 110 |
| 17 | Magnesium Fluoride (1261° C.) | Semi Solid 98 |
| | Sulphates | |
| 18 | Sodium Sulphate (884° C.) | Semi Solid at 90 |
| 19 | Zinc Sulphate (100° C.) | Semi Solid at 90 |
| 20 | Aluminium Sulphate (86.5° C.) | Semi Solid at 76 |
| 21 | Ammonium Ferric Sulphate (41° C.) | Semi Solid at 30 |
| 22 | Magnesium Sulphate (150° C.) | Semi Solid at 20 |
| 23 | Calcium Sulphate (1450° C.) | Semi Solid at 71 |
| 24 | Ferrous Sulphate (70° C.) | Semi Solid at 56 |
| 25 | Cupric Sulphate (150° C.) | Semi Solid at 71 |
| 26 | Nickel Sulphate (53° C.) | Semi Solid at 69 |
| 27 | Potassium Sulphate (1069° C.) | Semi Solid at 99 |
| | Nitrates | |
| 28 | Sodium Nitrate (308° C.) | Semi Solid at 72 |
| 29 | Aluminium Nitrate (73° C.) | Semi Solid at 3 8 |
| 30 | Ammonium Nitrate (170° C.) | Semi Solid at 71 |
| 31 | Potassium Nitrate (334° C.) | Semi Solid at 80 |
| 32 | Nickel Nitrate (57° C.) | Semi Solid at 21 |
| | Bromides | |
| 33 | Potassium Bromide (734° C.) | Semi Solid at 91 |
| 34 | Cobalt Bromide (678° C.) | Semi Solid at 56 |
| 35 | Cetylpyridinum Bromide (70° C.) | Semi Solid at 61 |
| 36 | Lithium Bromide (552° C.) | Semi Solid at 121 |
| | Acetates | |
| 37 | Sodium Acetate (324° C.) | Semi Solid at 20 |
| 38 | Zinc Acetate (237° C.) | Semi Solid at 21 |
| 39 | Ammonium Acetate (114° C.) | Semi Solid at 20 |
| 40 | Cobalt Acetate (140° C.) | Semi Solid at 49 |
| 41 | Manganese Acetate (210° C.) | Semi Solid at 51 |
| 42 | Lead Acetate (280° C.) | Semi Solid at 21 |

### EXAMPLES 43 TO 85

The procedure of example 1 was followed except that oxalic acid was used instead of p-Toluenesulfonic acid (refer the Table 2 below).

TABLE 2
Hydrogen Donor: Oxalic Acid

| | | State of resultant |
|---|---|---|
| Example | Salt (melting point ° C.) | precursor (at ° C.) |
| | Chlorides | |
| 43 | Sodium Chloride (801° C.) | Semi Solid at 89 |
| 44 | Zinc Chloride (292° C.) | Semi Solid at 24 |
| 45 | Ferric Chloride (306° C.) | Semi Solid at 23 |
| 46 | Cobaltous Chloride (735° C.) | Semi Solid at 54 |
| 47 | Cuprous Chloride (426° C.) | Semi Solid at 89 |
| 48 | Mangenous Chloride (58° C.) | Semi Solid at76 |
| 49 | Nickel Chloride (140° C.) | Semi Solid at 48 |
| 50 | Potassium Chloride (770° C.) | Semi Solid at 79 |
| 51 | Calcium Chloride (772° C.) | Semi Solid at 81 |
| 52 | Stannous Chloride (247° C.) | Semi Solid at 24 |
| 53 | Cesium Chloride (645° C.) | Semi Solid at 51 |
| 54 | Magnesium Chloride (714° C.) | Semi Solid at 22 |
| 55 | Mercury Chloride (276° C.) | Semi Solid at 99 |
| 56 | Choline Chloride (302° C.) | Liquid at 20 |
| | Fluorides | |
| 57 | Sodium Fluoride (993° C.) | Semi Solid at 79 |
| 58 | Calcium Fluoride (1418° C.) | Semi Solid at 101 |
| 59 | Potassium Fluoride (858° C.) | Semi Solid at 64 |
| 60 | Magnesium Fluoride (1261° C.) | Semi Solid at 109 |
| | Sulphates | |
| 61 | Sodium Sulphate (884° C.) | Semi Solid at 81 |
| 62 | Zinc Sulphate (100° C.) | Semi Solid at 19 |
| 63 | Aluminium Sulphate (87° C.) | Semi Solid at 54 |
| 64 | Ammonium Ferric Sulphate (41° C.) | Semi Solid at 18 |
| 65 | Magnesium Sulphate (150° C.) | Semi Solid at 73 |
| 66 | Calcium Sulphate (1450° C.) | Semi Solid at 104 |
| 67 | Ferrous Sulphate (70° C.) | Semi Solid at 28 |
| 68 | Cupric Sulphate (150° C.) | Semi Solid at 21 |
| 69 | Nickel Sulphate (53° C.) | Semi Solid at 36 |
| 70 | Potassium Sulphate (1069° C.) | Semi Solid at 68 |
| | Nitrates | |
| 71 | Sodium Nitrate (308° C.) | Semi Solid at 66 |
| 72 | Aluminium Nitrate (73° C.) | Semi Solid at 28 |
| 73 | Ammonium Nitrate (170° C.) | Semi Solid at 49 |
| 74 | Potassium Nitrate (334° C.) | Semi Solid at 56 |
| 75 | Nickel Nitrate (57° C.) | Semi Solid at 54 |
| | Bromides | |
| 76 | Potassium Bromide (734° C.) | Semi Solid at 79 |
| 77 | Cobalt Bromide (678° C.) | Semi Solid at 48 |
| 78 | Cetylpyridinum Bromide (70° C.) | Semi Solid at 78 |
| 79 | Lithium Bromide (552° C.) | Semi Solid at 22 |
| | Acetates | |
| 80 | Sodium Acetate (324° C.) | Semi Solid at 21 |
| 81 | Zinc Acetate (237° C.) | Semi Solid at 23 |
| 82 | Ammonium Acetate (114° C.) | Semi Solid at 24 |
| 83 | Cobalt Acetate (140° C.) | Semi Solid at 59 |
| 84 | Manganese Acetate (210° C.) | Semi Solid at 74 |
| 85 | Lead Acetate (280° C.) | Semi Solid at 49 |

### EXAMPLES 86 TO 124

The procedure of example 1 was followed except that maleic acid was used instead of p-Toluenesulfonic acid (refer the Table 3 below).

TABLE 3
Hydrogen Donor: Maleic acid

| | | State of resultant |
|---|---|---|
| Example | Salt (melting point ° C.) | precursor (at ° C.) |
| | Chlorides | |
| 86 | Sodium Chloride (801° C.) | Semi Solid at 99 |
| 87 | Zinc Chloride (292° C.) | Semi Solid at 101 |
| 88 | Ferric Chloride (306° C.) | Semi Solid at 25 |
| 89 | Cobaltous Chloride (735° C.) | Semi Solid at 79 |
| 90 | Cuprous Chloride (426° C.) | Semi Solid at 111 |
| 91 | Mangenous Chloride (58° C.) | Semi Solid at 116 |
| 92 | Nickel Chloride (140° C.) | Semi Solid at 105 |
| 93 | Potassium Chloride (770° C.) | Semi Solid at 98 |
| 94 | Calcium Chloride (772° C.) | Semi Solid at 101 |
| 95 | Stannous Chloride (247° C.) | Semi Solid at 84 |
| 96 | Magnesium Chloride (714° C.) | Semi Solid at 93 |
| 97 | Mercury Chloride (276° C.) | Semi Solid at 141 |
| 98 | Choline Chloride (302° C.) | Liquid at 10 |
| | Fluorides | |
| 99 | Sodium Fluoride (993° C.) | Semi Solid at 102 |
| 100 | Potassium Fluoride (858° C.) | Semi Solid at 108 |
| 101 | Magnesium Fluoride (1216° C.) | Semi Solid at 96 |
| | Sulphates | |
| 102 | Sodium Sulphate (884° C.) | Semi Solid at 134 |
| 103 | Zinc Sulphate (100° C.) | Semi Solid at 86 |
| 104 | Ammonium Ferric Sulphate (47° C.) | Semi Solid at 50 |
| 105 | Magnesium Sulphate (150° C.) | Semi Solid at 98 |
| 106 | Calcium Sulphate (1450° C.) | Semi Solid at 100 |
| 107 | Cupric Sulphate (150° C.) | Semi Solid at 121 |
| 108 | Nickel Sulphate (53° C.) | Semi Solid at 130 |
| 109 | Potassium Sulphate (1069° C.) | Semi Solid at 128 |
| | Nitrates | |
| 110 | Sodium Nitrate (308° C.) | Semi Solid at 121 |
| 111 | Aluminium Nitrate (73° C.) | Semi Solid at 76 |
| 112 | Ammonium Nitrate (170° C.) | Semi Solid at 74 |
| 113 | Potassium Nitrate (334° C.) | Semi Solid at 120 |
| 114 | Nickel Nitrate (57° C.) | Semi Solid at 48 |
| | Bromides | |
| 115 | Potassium Bromide (734° C.) | Semi Solid at 129 |
| 116 | Cobalt Bromide (678° C.) | Semi Solid at 48 |
| 117 | Cetylpyridinum Bromide (70° C.) | Semi Solid at 39 |
| 118 | Lithium Bromide (552° C.) | Semi Solid at 61 |
| | Acetates | |
| 119 | Sodium Acetate (324° C.) | Semi Solid at 49 |
| 120 | Zinc Acetate (237° C.) | Semi Solid at 119 |
| 121 | Ammonium Acetate (114° C.) | Semi Solid at 54 |
| 122 | Cobalt Acetate (140° C.) | Semi Solid at 59 |
| 123 | Manganese Acetate (210° C.) | Semi Solid at 57 |
| 124 | Lead Acetate (280° C.) | Semi Solid at 55 |

### EXAMPLES 125 TO 167

The procedure of example 1 was followed except that citric acid was used instead of p-Toluenesulfonic acid (refer the Table 4 below).

TABLE 4
Hydrogen Donor: Citric Acid

| | | State of resultant |
|---|---|---|
| Example | Salt (melting point ° C.) | precursor (at ° C.) |
| | Chlorides | |
| 125 | Zinc Chloride (292° C.) | Semi Solid at 20 |
| 126 | Sodium Chloride (801° C.) | Semi Solid at 49 |
| 127 | Ferric Chloride (306° C.) | Semi Solid at 23 |
| 128 | Cobaltous Chloride (735° C.) | Semi Solid at 69 |
| 129 | Cuprous Chloride (426° C.) | Semi Solid at 91 |
| 130 | Mangenous Chloride (58° C.) | Semi Solid at 64 |
| 131 | Nickel Chloride (140° C.) | Semi Solid at 49 |
| 132 | Potassium Chloride (770° C.) | Semi Solid at 51 |
| 133 | Calcium Chloride (772° C.) | Semi Solid at 56 |
| 134 | Stannous Chloride (247° C.) | Semi Solid at 49 |
| 135 | Cesium Chloride (645° C.) | Semi Solid at 29 |
| 136 | Magnesium Chloride (714° C.) | Semi Solid at 98 |
| 137 | Mercury Chloride (276° C.) | Semi Solid at 54 |
| 138 | Choline Chloride (302° C.) | Semi Solid at 35 |
| | Fluorides | |
| 139 | Sodium Fluoride (993° C.) | Semi Solid at 89 |
| 140 | Calcium Fluoride (1418° C.) | Semi Solid at 101 |
| 141 | Potassium Fluoride (858° C.) | Semi Solid at 90 |
| 142 | Magnesium Fluoride (1261° C.) | Semi Solid at 58 |
| | Sulphates | |
| 143 | Sodium Sulphate (884° C.) | Semi Solid at 63 |
| 144 | Zinc Sulphate (100° C.) | Semi Solid at 72 |
| 145 | Aluminium Sulphate (87° C.) | Semi Solid at 93 |
| 146 | Ammonium Ferric Sulphate (41° C.) | Semi Solid at 44 |
| 147 | Magnesium Sulphate (150° C.) | Semi Solid at 69 |
| 148 | Calcium Sulphate (1450° C.) | Semi Solid at 99 |
| 149 | Ferrous Sulphate (70° C.) | Semi Solid at 59 |
| 150 | Cupric Sulphate (150° C.) | Semi Solid at 73 |
| 151 | Nickel Sulphate (53° C.) | Semi Solid at 38 |
| 152 | Potassium Sulphate (1069° C.) | Semi Solid at 76 |
| | Nitrates | |
| 153 | Sodium Nitrate (308° C.) | Semi Solid at 54 |
| 154 | Aluminium Nitrate (73° C.) | Semi Solid at 49 |
| 155 | Ammonium Nitrate (170° C.) | Semi Solid at 22 |
| 156 | Potassium Nitrate (334° C.) | Semi Solid at 73 |
| 157 | Nickel Nitrate (57° C.) | Semi Solid at 52 |
| | Bromides | |
| 158 | Potassium Bromide (734° C.) | Semi Solid at 54 |
| 159 | Cobalt Bromide (678° C.) | Semi Solid at 59 |
| 160 | Cetylpyridinum Bromide (70° C.) | Semi Solid at 72 |
| 161 | Lithium Bromide (552° C.) | Semi Solid at 24 |
| | Acetates | |
| 162 | Sodium Acetate (324° C.) | Semi Solid at 21 |
| 163 | Zinc Acetate (237° C.) | Semi Solid at 59 |
| 164 | Ammonium Acetate (114° C.) | Semi Solid at 22 |
| 165 | Cobalt Acetate (140° C.) | Semi Solid at 58 |
| 166 | Manganese Acetate (210° C.) | Semi Solid at 59 |
| 167 | Lead Acetate (280° C.) | Semi Solid at 58 |

### EXAMPLES 168 TO 206

The procedure of example 1 was followed except that methane sulfonic was used instead of p-Toluenesulfonic acid (refer the Table 5 below).

TABLE 5
Hydrogen Donor: Methane sulfonicacid

| | | State of the resultant |
|---|---|---|
| Example | Salt (melting point ° C.) | precursor (at ° C.) |
| | Chlorides | |
| 168 | Zinc Chloride (292° C.) | Semi Solid at 22 |
| 169 | Sodium Chloride (801° C.) | Semi Solid at 22 |
| 170 | Ferric Chloride (306° C.) | Semi Solid at 22 |
| 171 | Cobaltous Chloride (735° C.) | Semi Solid at 22 |
| 172 | Cuprous Chloride (426° C.) | Semi Solid at 22 |
| 173 | Mangenous Chloride (58° C.) | Semi Solid at 22 |
| 174 | Nickel Chloride (140° C.) | Semi Solid at 22 |
| 175 | Potassium Chloride (770° C.) | Semi Solid at 22 |
| 176 | Calcium Chloride (772° C.) | Semi Solid at 22 |
| 177 | Stannous Chloride (247° C.) | Semi Solid at 22 |
| 178 | Magnesium Chloride (714° C.) | Semi Solid at 22 |
| 179 | Mercury Chloride (276° C.) | Semi Solid at 22 |
| 180 | Choline Chloride (302° C.) | Liquid at 0 |
| | Fluorides | |
| 181 | Sodium Fluoride (993° C.) | Semi Solid at 22 |
| 182 | Calcium Fluoride (1418° C.) | Semi Solid at 22 |
| 183 | Potassium Fluoride (858° C.) | Semi Solid at 22 |
| 184 | Magnesium Fluoride (1261° C.) | Semi Solid at 22 |
| | Sulphates | |
| 185 | Sodium Sulphate (884° C.) | Semi Solid at 22 |
| 186 | Zinc Sulphate (100° C.) | Semi Solid at 22 |
| 187 | Ammonium Ferric Sulphate (41° C.) | Semi Solid at 22 |
| 188 | Magnesium Sulphate (150° C.) | Semi Solid at 22 |
| 189 | Calcium Sulphate (1450° C.) | Semi Solid at 22 |
| 190 | Cupric Sulphate (150° C.) | Semi Solid at 22 |
| 191 | Nickel Sulphate (53° C.) | Semi Solid at 22 |
| 192 | Potassium Sulphate (1069° C.) | Semi Solid at 22 |
| | Nitrates | |
| 193 | Sodium Nitrate (308° C.) | Semi Solid at 22 |
| 194 | Aluminium Nitrate (73° C.) | Semi Solid at 22 |
| 195 | Ammonium Nitrate (170° C.) | Semi Solid at 22 |
| 196 | Potassium Nitrate (334° C.) | Semi Solid at 22 |
| 197 | Nickel Nitrate (57° C.) | Semi Solid at 22 |
| | Bromides | |
| 198 | Potassium Bromide (734° C.) | Semi Solid at 22 |
| 199 | Cobalt Bromide (678° C.) | Semi Solid at 22 |
| 200 | Cetylpyridinum Bromide (70° C.) | Semi Solid at 22 |
| 201 | Lithium Bromide (552° C.) | Semi Solid at 22 |
| | Acetates | |
| 202 | Sodium Acetate (324° C.) | Semi Solid at 22 |
| 203 | Zinc Acetate (237° C.) | Semi Solid at 22 |
| 204 | Ammonium Acetate (114° C.) | Semi Solid at 22 |
| 205 | Cobalt Acetate (140° C.) | Semi Solid at 22 |
| 206 | Lead Acetate (280° C.) | Semi Solid at 22 |

### EXAMPLES 207-408

### Preparation of Ionic Fluid

The procedure of example 3 was followed to prepare ionic fluid from the Ionic fluid precursors of examples 5-206.

Solvents employed for the preparation of ionic fluid are as follows:
Examples 207 to 244—methanol,
Examples 245 to 287—water,
Examples 288 to 326—dimethyl formamide
Examples 327 to 369—acetic acid
Examples 370 to 408—ethylene glycol

### EXAMPLE 409

### Preparation of Ionic Fluid

0.9 kilograms of oxalic acid and 1.36 kilograms of zinc chloride were charged into different hoppers. From the hoppers both the solids were passed through a screw mixer and simultaneously 0.09 kilograms of methanol was also introduced to the screw mixer from another vessel to a planetary mixer at 80 rpm to form in-situ ionic fluid at 28° C.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Technical Advancement and Economic Significance
The present disclosure provides preparation of ionic fluid pre-cursor at low temperature i.e. 0 to 40° C.
The present disclosure provides preparation of ionic fluid pre-cursor without employing any liquid medium.
The present disclosure provides preparation of ionic fluid pre-cursor using mechanical means such as mixer, thus energy input is not through heat and hence the process is a low temperature process.
The present disclosure provides an ionic fluid pre-cursor which is not a mere mixture and has different physical characteristic features from both its constituents, viz., Compound (I) and hydrogen donor compound, and is shelf stable.
The present disclosure also provides a method for preparation of an ionic fluid using a very low amount of liquid medium [0.1 wt % w.r.t compound of formula (I)] at a temperature of 0 to 40° C. without employing heat.
There is no loss of ionic strength by acid fume liberation during preparation and shelf life of said ionic fluid precursor and also while converting to the respective ionic fluid by assistance of a liquid medium
There is no salt formation and hence no requirement of filtration.
Liquid medium can be added for the benefit of deployment in reactions. e.g. for making the ionic fluid pre-cursor low viscous, breaking the gel nature of the ionic fluid pre-cursor and the like.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the invention to achieve one or more of the desired objects or results.

The numerical values given for various physical parameters, dimensions and quantities are only approximate values and it is envisaged that the values higher than the numerical value assigned to the physical parameters, dimensions and quantities fall within the scope of the invention and the claims unless there is a statement in the specification to the contrary.

While certain embodiments of the inventions have been described, these embodiments have been presented by way of example only, and are not intended to limit the scope of the inventions. Variations or modifications in the process or compound or formulation or combination of this invention, within the scope of the invention, may occur to those skilled in the art upon reviewing the disclosure herein. Such variations or modifications are well within the spirit of this invention. The accompanying claims and their equivalents are intended to cover such forms or modifications as would fall within the scope and spirit of the invention.

What is claimed is:

1. An ionic fluid pre-cursor, being a reaction product of at least one compound of formula (I) and at least one hydrogen donor and having a softening point less than the melting point or softening point of said compound of formula (I)
MₓA_{y}.zH₂O Formula (I)
wherein,
M is independently selected from the group consisting of Na, K, Li, Mg, Ca, Cr, Mn, Fe, Co, Mo, Ni, Cu, Zn, Cd, Sn, Pb, St, Bi, La, Ce, Al, Hg, Cs, Rb, Sr, V, Pd, Zr, Au, Pt, quarternary ammonium, immidazolium, phosphonium, pyridinium and pyrrolidinium,
A is independently selected from the group consisting of Cl, Br, F, I, NO₃, SO₄, CH₃COO, HCOO and C₂O₄,
z is 0 to 20, and
x and y are integers independently ranging from 1 to 20.

2. The ionic fluid pre-cursor as claimed in claim 1, wherein said precursor is maintained at a temperature of not more than 40° C.

3. The ionic fluid pre-cursor as claimed in claim 1, wherein during the preparation or storage of said ionic liquid pre-cursor and its conversion to ionic fluid, acidic fumes are not liberated in the form of compound of formula HₓAy.

4. The ionic fluid pre-cursor as claimed in claim 1, characterized in that said ionic fluid pre-cursor is adapted to convert into ionic fluid without precipitation of salt.

5. The ionic fluid pre-cursor as claimed in claim 1, wherein said hydrogen donor is at least one compound selected from the group consisting of toluene-4-sulphonic acid monohydrate, oxalic acid, maleic acid, citric acid and methane sulfonic acid.

6. The ionic fluid pre-cursor as claimed in claim 1, wherein the molar ratio of compound of formula (I) to said hydrogen donor ranges from 1:1 to 1:6.

7. The ionic fluid pre-cursor as claimed in claim 1, wherein said ionic fluid precursor is capable of delivering a clear liquid when deployed as a constituent of a mixture comprising said ionic fluid precursor and at least one liquid medium and maintained at a temperature in the range of 0° C. to 40° C.

8. An ionic fluid comprising:
a) an ionic fluid pre-cursor being a reaction product of at least one compound of formula (I) and at least one hydrogen donor and having a softening point less than the melting point or softening point of said compound of formula (I)
MₓA_{y}.zH₂O Formula (I)
wherein,
M is independently selected from the group consisting of Na, K, Li, Mg, Ca, Cr, Mn, Fe, Co, Mo, Ni, Cu, Zn, Cd, Sn, Pb, St, Bi, La, Ce, Al, Hg, Cs, Rb, Sr, V, Pd, Zr, Au, Pt, quarternary ammonium, immidazolium, phosphonium, pyridinium and pyrrolidinium,
A is independently selected from the group consisting of Cl, Br, F, I, NO₃, SO₄, CH₃COO, HCOO and C₂O₄,
z is 0 to 20, and
x and y are integers independently ranging from 1 to 20; and
b) at least one liquid medium.

9. The ionic fluid as claimed in claim 8, wherein the liquid medium is selected from the group consisting of methanol, ethanol, propan-1-ol, propan-2-ol, 1-butanol, isobutanol, 2-butanol, tert-butanol, dichloromethane, tetrahydrofuran, methyl acetate, ethyl acetate, acetone, dimethylformamide, acetonitrile, dimethyl sulfoxide, formic acid, acetic acid, methyl ethyl ketone, dimethyl carbonate, diethyl ketone, acetic anhydride, acetone, tert-butyl methyl ether, diethyl amine, diethylene glycol, N,N-dimethylacetamide, diethylene glycol dimethyl ether, ethylene glycol dimethyl ether, ethylene glycol, glycerin, hexamethylphosphor amide, hexamethylphosphorous triamide, isoamyl alcohol, 2-methoxyethanol, 2-methoxyethyl acetate, 1-methyl-2-pyrrolidinone, nitromethane, propanoic acid, pyridine, hydrogen fluoride, hydrogen chloride and water.

10. The ionic fluid as claimed in claim 8, wherein the molar ratio of compound of formula (I) to said hydrogen donor ranges from 1:1 to 1:6.

11. The ionic fluid as claimed in claim 8, wherein the weight ratio of the ionic fluid precursor to said liquid medium ranges from 1:0.1 to 1:50.

12. The ionic fluid as claimed in claim 8, wherein the amount of the medium ranges from 1 to 30% of the total weight of the compound of formula (I) and hydrogen donor.

13. A process for the preparation of an ionic fluid precursor having a softening point less than the melting point or softening point of said compound of formula (I); said process comprising mixing at least one compound of formula MₓA_{y}.zH₂O (I) at a pre-determined proportion with at least one hydrogen donor at a temperature in the range of 0° C. to 40° C., to obtain the ionic fluid precursor;
wherein,
M is independently selected from the group consisting of Na, K, Li, Mg, Ca, Cr, Mn, Fe, Co, Mo, Ni, Cu, Zn, Cd, Sn, Pb, St, Bi, La, Ce, Al, Hg, Cs, Rb, Sr, V, Pd, Zr, Au, Pt, quarternary ammonium, immidazolium, phosphonium, pyridinium and pyrrolidinium;
A is independently selected from the group consisting of Cl, Br, F, I, NO₃, SO₄, CH₃COO, HCOO and C₂O₄,
z is 0 to 20; and
x and y are integers independently ranging from 1 to 20,
wherein said ionic fluid precursor is capable of delivering a clear liquid when deployed as a constituent of a mixture comprising said ionic fluid precursor and at least one liquid medium and maintained at a temperature in the range of 0° C. to 40° C.

14. The process as claimed in claim 13, wherein the liquid medium is selected from the group consisting of methanol, ethanol, propan-1-ol, propan-2-ol, 1-butanol, isobutanol, 2-butanol, tert-butanol, dichloromethane, tetrahydrofuran, methyl acetate, ethyl acetate, acetone, dimethylformamide, acetonitrile, dimethyl sulfoxide, formic acid, acetic acid, methyl ethyl ketone, dimethyl carbonate, diethyl ketone, acetic anhydride, acetone, tert-butyl methyl ether, diethyl amine, diethylene glycol, N,N-dimethylacetamide, diethylene glycol dimethyl ether, ethylene glycol dimethyl ether, ethylene glycol, glycerin, hexamethylphosphor amide, hexamethylphosphorous triamide, isoamyl alcohol, 2-methoxyethanol, 2-methoxyethyl acetate, 1-methyl-2-pyrrolidinone, nitromethane, propanoic acid, pyridine, hydrogen fluoride, hydrogen chloride and water.

15. The process as claimed in claim 13, wherein the hydrogen donor is at least one compound selected from the group consisting of toluene-4-sulphonic acid monohydrate, oxalic acid, maleic acid, citric acid and methane sulfonic acid.

16. The process as claimed in claim 13, wherein the molar ratio of compound of formula (I) to said hydrogen donor ranges from 1:1 to 1:6.
